(19) United States
(12) Patent Application Publication
(10) Pub. No.: US 2026/0272720 A1
(43) Pub. Date: Sep. 17, 2026

(54) WOUND CLEANSING SYSTEM

(71) Applicant: Brian Mark Dane, Sr., Newark, CA (US)

(72) Inventor: Brian Mark Dane, Sr., Newark, CA (US)

(21) Appl. No.: 19/082,078

(22) Filed: Mar. 17, 2025

(51) Int. Cl. A61F 13/05; A61F 13/00; A61F 13/02; A61M 1/00

(52) U.S. Cl. CPC A61F 13/05; A61F 13/02; A61M 1/85; A61F 2013/00536; A61F 2013/00846; A61F 2013/00889; A61F 2013/00978

(57) ABSTRACT

The present invention offers an efficient way to care for a patient's wounds. It allows for easy gauze changes without irritating a wound. The draining tube is used as a channel to deliver fluid to a wound. Infection can be monitored through the gauze as the cover component may include transparent regions. A wound can therefore be inspected by removing the cover component rather than an entire adhesive bandage. A wound can be kept clean, with quick and simple gauze changes several times a day thereby accelerating the healing process.

### FIELD

The present invention relates to caring for a patient's wound, particularly through the use of a novel cleansing apparatus.

### BACKGROUND

Cleaning a wound covered by an adhesive bandage presents several challenges that impact the wound healing process. An adhesive bandage is configured to remain securely affixed to the skin, providing a barrier against contaminants, moisture, and mechanical forces. However, improper removal of the bandage may result in pain, irritation to the surrounding skin, or detachment of newly formed tissue, thereby potentially delaying wound healing. Additionally, adhesion of the bandage to the wound or an associated scab may cause difficulty in removal, increasing the risk of trauma to the wound site.

A further challenge pertains to maintaining sterility during the wound-cleansing process. Upon removal of the adhesive bandage, a wound is exposed to ambient air and external contaminants, thereby increasing the likelihood of bacterial infiltration or particulate contamination. Residual adhesive from the bandage may remain on the skin or wound site, complicating the cleansing process. Moreover, wound cleansing necessitates careful handling to mitigate irritation of the tissue, as excessive mechanical disruption may induce swelling or increase the risk of infection. Furthermore, application of an inappropriate cleansing agent, such as a solution containing harsh chemical compounds, may result in additional damage to the regenerating skin.

The timing of adhesive bandage removal and wound cleansing is also a critical consideration. Prolonged retention of the bandage may create a localized warm and moist environment conducive to bacterial proliferation. Conversely, premature removal of the bandage may expose the wound to external contaminants before sufficient healing has occurred.

Accordingly, an optimal balance must be maintained between protecting the wound and ensuring appropriate cleansing procedures to mitigate infection risks. Effective wound management necessitates an approach that accounts for wound condition, proper bandage removal techniques, and selection of suitable cleansing methods.

U.S. Patent Publication No. 2014/0296813A1 teaches a pillow-shaped wound care article for the extraction and control of wound fluids. This type of wound care article is especially suitable for the extraction of exudate from chronic wounds, such as (for example) when diabetes, ulcus cruris, and similar illnesses occur.

U.S. Pat. No. 8,377,015B2 teaches a wound dressing that comprises a perforated material having a top face and a bottom face and having plural through-holes, and an absorbent material disposed on the top face of the perforated material, wherein at least the bottom face of the perforated material is coated with a low-adhesive resin except leaving at least a part of the through-holes so as not to block up all the through-holes, and at least a part of the absorbent material facing the through-holes of the perforated material is coated with the low-adhesive resin, and to allow fluid penetration from the bottom face side of the perforated material into the inside of the absorbent material. Preferably, the top face of the perforated material is coated with the low-adhesive resin except leaving at least a part of the through-holes so as not to block up all the through-holes.

U.S. Patent Publication No. 2011/0004172A1 teaches a new device for vacuum therapy of wounds comprising a cover material that is impermeable to air for air-tight closure of the wound and the wound environment, and a means for functional connection of the wound space with a source of vacuum outside the cover material so that a vacuum can be established in the wound space and liquids can be sucked out of the wound space.

U.S. Pat. No. 11,617,823 teaches an apparatus for cleansing wounds with means for stressing the wound bed and optionally tissue surrounding the wound, in which irrigant fluid from a reservoir connected to a conformable wound dressing and wound exudate from the dressing are recirculated by a devise for moving fluid through a flow path which passes through the dressing and a means for fluid cleansing and back to the dressing. The cleansing means (which may be a single-phase, e.g. micro-filtration, system or a two-phase, e.g. dialytic system) removes materials deleterious to wound healing, and the cleansed fluid, still containing materials that are beneficial in promoting wound healing, is returned to the wound bed. The means for stressing the wound bed and optionally tissue surrounding the wound promotes wound healing.

A need exists for an effective solution to care for a patient's wound that overcomes the limitations of prior approaches. The present invention solves this need.

### BRIEF DESCRIPTION OF THE DRAWINGS

To facilitate understanding, identical reference numerals have been used, wherever possible, to designate identical elements that are common to the figures. The drawings are not to scale and the relative dimensions of various elements in the drawings are depicted schematically and not necessarily to scale. The techniques of the present disclosure may readily be understood by considering the following detailed description in conjunction with the accompanying drawings.

FIG. 1 is an illustration of a wound cleansing apparatus according to one embodiment of the present invention.

FIG. 2A-2E illustrate a wound cleansing apparatus throughout the process of replacing a set of gauzes.

FIG. 3 illustrates a wound cleansing method according to an embodiment of the present invention.

FIG. 4 illustrates a method for replacing gauzes in a wound cleaning apparatus according to an embodiment of the present invention.

FIG. 5 illustrates a wound cleansing system according to an embodiment of the present invention.

### DETAILED DESCRIPTION

Before the present disclosure is described in detail, it is to be understood that, unless otherwise indicated, this disclosure is not limited to specific procedures or articles, whether described or not. It is further to be understood that the terminology used herein is for the purpose of describing particular embodiments only and is not intended to limit the scope of the present disclosure. It must be noted that as used herein, and in the claims, the singular forms “a,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. The term “about” generally refers to ±10% of a stated value.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

This specification includes references to “one embodiment” or “an embodiment. The appearances of the phrases “in one embodiment” or “in an embodiment” do not necessarily refer to the same embodiment. Particular features, structures, or characteristics may be combined in any suitable manner consistent with this disclosure.

Terminology. The following paragraphs provide definitions and/or context For terms found in this disclosure (including the appended claims):

“Comprising.” This term is open-ended. As used in the appended claims, this term does not foreclose additional structure or steps.

“Configured To.” Various units or components may be described or claimed as “configured to: perform a task or tasks. In such contexts, “configured to” is used to connote structure by indicating that the units/components include structure that performs those task or tasks during operation. As such, the unit/component can be said to be configured to perform the task even when the specified unit/component is not currently operational (e.g., is not on/active). Reciting that a unit/circuit/component is “configured to” perform one or more tasks is expressly intended not to invoke 35 U.S.C. § 112, sixth paragraph, for that unit/component. “First,” “Second,” etc. As used herein, these terms are used as labels for nouns that they precede, and do not imply any type of ordering (e.g., spatial, temporal, logical, etc.). For example, reference to a “first” pre-defined distance does not necessarily imply that this pre-defined distance is the first pre-defined distance in a sequence; instead, the term “first” is used to differentiate this pre-defined distance from another pre-defined distance (e.g., a “second” pre-defined distance).

The invention offers an efficient way to care for patient wounds with drain tubes. It allows for easy gauze changes without removing the adhesive bandage, and the adhesive bandage is waterproof, enabling the wearer to shower while keeping it on. The draining tube can be flushed, allowing visibility of what is being flushed out of the wound, and the gauzes can be changed simply by removing the cover. Infection can be monitored through the gauze, and the draining tube stays securely locked in place to prevent movement. A patient's wound can be inspected by removing the cover rather than the entire adhesive bandage. Therefore, the wound can be kept clean, with quick and simple gauze changes several times a day thereby accelerating the healing process.

An object of the present invention is a wound cleansing apparatus which includes a cover component that connects to a base component by a coupling means. In one implementation, the cover and base components are attached together by a snap connection. Inside the base component is at least one piece of gauze. The base component also has a draining tube attached to it using a latching mechanism.

Another object of the present invention is a wound cleansing system which includes a wound cleansing apparatus that was previously described. The wound cleansing system further includes a fluid dispenser coupled to an end of the draining tube to provide a fluid for cleansing a wound.

A further object of present invention is a wound cleansing method. The method begins with applying a wound cleansing system to a patient's wound. The system includes a wound cleansing apparatus with a cover component coupled to a base component with a draining tube attached therewith by a latching mechanism. Within an interior of the base component is a set of gauzes which contact the wound. To clean the wound, a fluid is injected into the draining tube which leads the fluid to the wound.

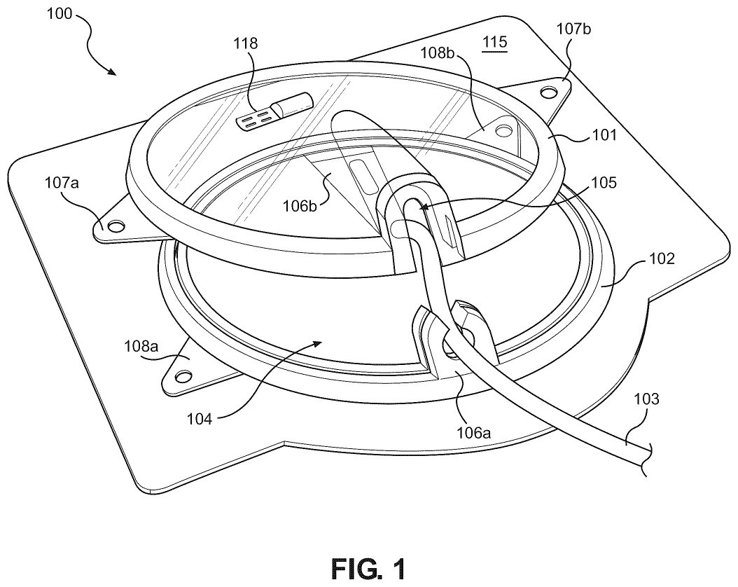

FIG. 1 is an illustration of a wound cleansing apparatus 100 according to one embodiment of the present invention. The wound cleansing apparatus 100 includes cover component 101, base component 102, and draining tube 103. In some implementations, the wound cleansing apparatus 100 has an adhesive bandage 115 extending from the base component 102.

In the embodiment shown in the figure, the cover component 101 includes a body with a bend 105, an air vent 118, flange components 107a, 107b, and snap connection components (not shown). In some implementations, the cover component 101 is a female snap-on ring cover component 101 which is designed to mate with a base component 102. In some implementations, base component 102 is a male snap-on ring base component 102.

The cover component 101 helps protect a patient's wound from the environment. The cover component 101 includes a transparent portion such that a patient's wound is visible through the cover component 101. The cover component 101 may comprise plastic and therefore may have a flexible body.

The cover component 101 may include an air vent 118 that provides an opening to allow air to pass therethrough or into a confined space to allow a patient's wound to breathe. When the air vent 118 is in an open position, air is allowed to pass through and when the air vent 118 is in a closed position, air is prevented from reaching the wound. The cover component 101 may include a “bend” for the draining tube 103. In some implementations, the “bend” in the cover component may be in the range of 20%-40%.

In the figure shown, the cover component 101 includes a pair of flange components 107a, 107b. The pair of flange components 107a, 107b may aid in the alignment of cover component 101 with base component 102 during assembly. For example, the flange components 107a, 107b of cover component 101 may be aligned with the flange components 108a, 108b of base component 102.

When the wound cleansing apparatus 100 is assembled, the base component 102 is beneath the cover component 101. When the wound cleansing apparatus 100 is in operation, the base component 102 is proximate to the patient's wound.

In the embodiment shown in the figure, the base component 102 has a circular body and includes latching mechanism 106, flange components 108, and snap connection components (not shown).

In addition, a set of gauzes 104 are fitted within the interior of the male snap-on ring base component 102. Herein, a gauze is defined by a thin, translucent fabric with a loose open weave. The set of gauzes 104 may consist of one, two, or more gauzes fitted within the interior of the base component 102. In some implementations, when the wound cleansing apparatus 100 is in operation, the set of gauzes 104 is in direct contact with a patient's wound. It should be understood by those having ordinary skill in the art that the wound cleansing apparatus 100.

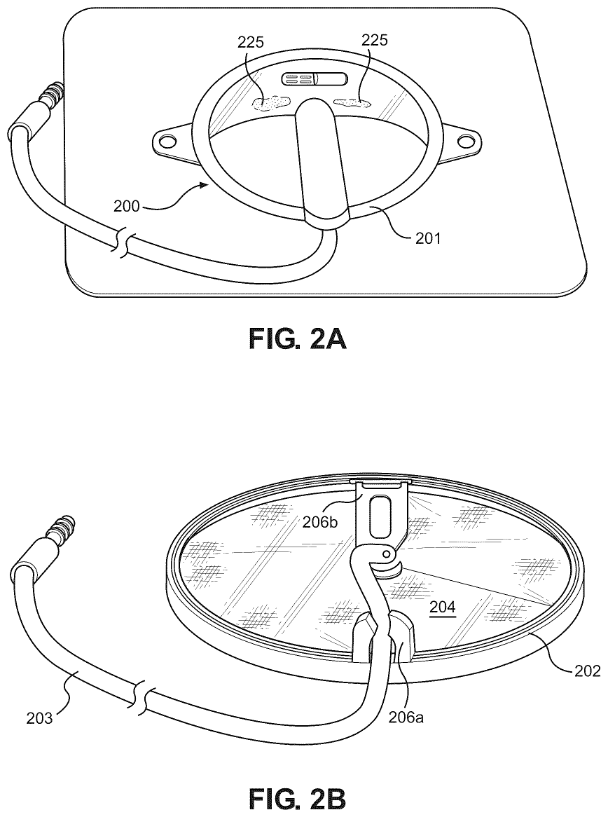

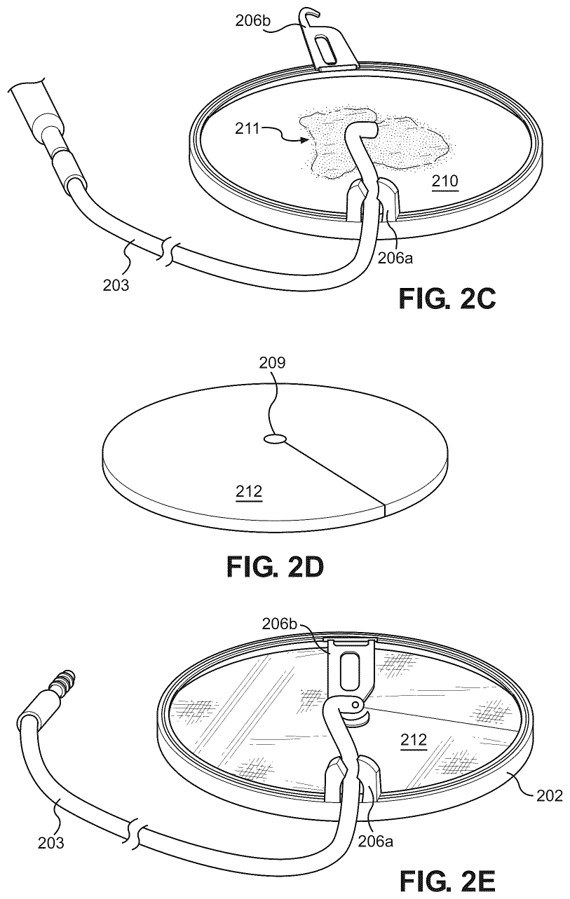

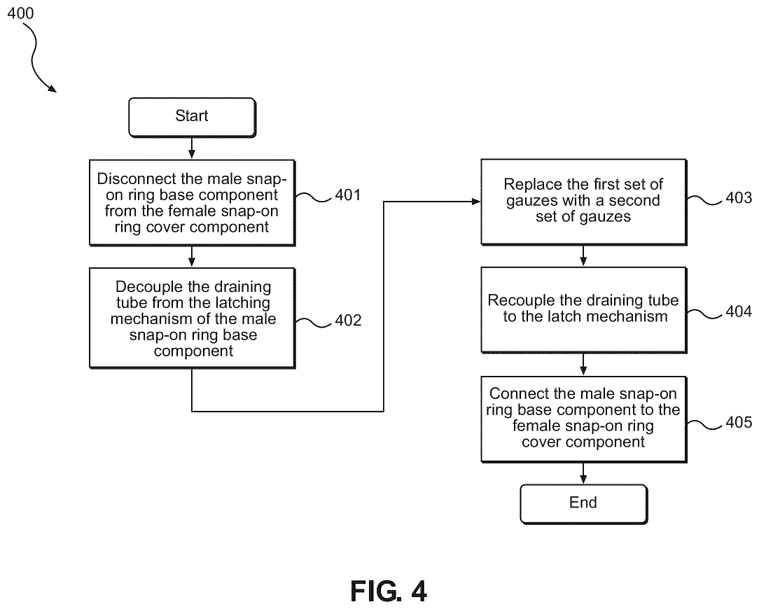

FIG. 2A-2E illustrate a wound cleansing apparatus 200 throughout the process of replacing the set of gauzes 204. The process of replacing gauzes in a wound cleansing apparatus 200 is further described in FIG. 4.

FIG. 2A illustrates a perspective view of a wound cleansing apparatus 200 before the set of gauzes 204 are replaced. In the figure, the wound cleansing apparatus 200 is fully assembled and therefore the female snap-on ring cover component 201 and the male snap-on ring base component 202 are coupled together.

FIG. 2B illustrates the male snap-on ring base component 202 with a set of gauzes 204 with the latching mechanism 206 disengaged. The male snap-on ring base component 202 has been disconnected from the female snap-on ring cover component 201 and removed from the wound cleansing apparatus 200. As further shown, the draining tube is locked in place by the latching mechanism 206 by latch components 206a, 206b. To replace the set of gauzes 204, the latching mechanism 206 must be first disengaged. However, before the latching mechanism 206 is disengaged, the draining tube 203 is locked in place by first latch component 206a and second latch component 206b.

FIG. 2C illustrates that the set of gauzes 204 have been removed from the male snap-on ring component 202 such that the wound 211 is exposed. As illustrated, because the latching mechanism 206 is disengaged, the latch component 206b is removed from the draining tube 203.

FIG. 2D illustrates a new set of gauzes 212 which can be fitted within the male snap-on ring base component 202 of the wound cleansing apparatus 200.

FIG. 2E illustrates the male snap-on ring base component 202 of the wound cleansing apparatus 200 after the new set of gauzes 212 are lodged in the interior of the male snap-on ring base component 202. Afterwards, the latching mechanism 206 can be re-engaged such that the draining tube 203 is locked in and set in position by latching components 206a, 206b.

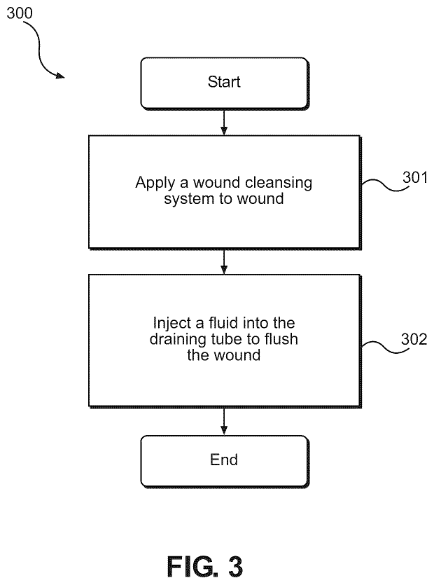

FIG. 3 illustrates a wound cleansing method 300 according to an embodiment of the present invention. Method 300 begins with applying a wound cleansing apparatus consistent with the present invention to a wound (block 301). Method 300 proceeds to inject a fluid into the draining tube to flush the wound (block 302). In some implementations, the fluid injected into the draining tube is saline. However, the present invention is not limited thereto. The fluid may consist of water, medicine in liquid form, or any other suitable fluid that aids in a healing process.

FIG. 4 illustrates a method 400 for replacing gauzes in a wound cleansing apparatus according to an embodiment of the present invention. Method 400 refers to the wound cleansing apparatus 200 illustrated in FIG. 2A-2E. Method 400 begins with disconnecting the male snap-on ring base component from the female snap-on ring cover component (block 401). Next, decouple the draining tube from the latching mechanism of the male snap-on ring base component (block 402). Afterwards, replace the first set of gauzes with a second set of gauzes (block 403). Once the new set of gauzes are installed within the male snap-on ring base component, recouple the draining tube to the latching mechanism (block 404). Lastly, connect the male snap-on ring base component to the female snap-on ring cover component (block 405).

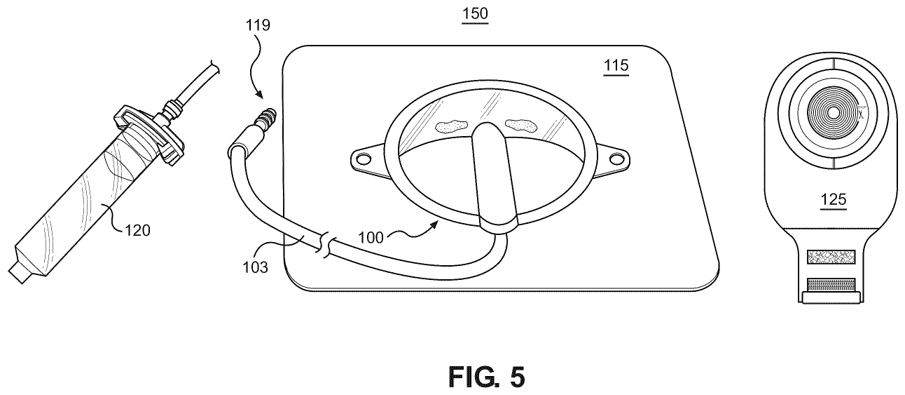

FIG. 5 illustrates a wound cleansing system 150 according to an embodiment of the present invention. In the embodiment shown in the figure, wound cleansing system 150 includes a wound cleansing apparatus 100, an adhesive bandage 115, a fluid dispenser 120, and a colostomy bag 125. However, one having ordinary skill in the art should appreciate that the wound cleansing system 150 may include all or less than the devices illustrated in FIG. 5 so long as the wound cleansing apparatus 100 is included therein. As such, a wound cleansing system 150 could only consist of a wound cleansing apparatus 100 and a fluid dispenser 120 in some implementations of the invention.

In the embodiment shown, an adhesive bandage 115 may be placed on the exterior of the wound cleansing apparatus 100. The adhesive bandage 115 may be waterproof. Adhesive bandage 115 protects the wound and scab from bacteria, damage, or dirt, so that the healing process of the body is less disturbed. Adhesive bandage 115 may comprise an acrylic polymer.

In the embodiment shown in FIG. 5, the fluid dispenser 120 is a positive displacement dispenser. Herein, a positive displacement dispenser is defined herein as a syringe style pump where when the pump piston moves, it moves through the pump chamber and positively displaces the fluid with the piston. The fluid moves as the piston displaces it.

The fluid dispenser 120 may be used to inject fluid into the draining tube 103 to flush and clean a wound. The fluid may be water (e.g., sterile water), saline, or other medicine in fluid form. The fluid dispenser 120 is coupled to one end 119 of the draining tube 103.

An optional colostomy bag 125 may be coupled to the wound cleansing apparatus 100 by any suitable coupling means when needed by a patient (e.g., after a colostomy). In some cases, after a surgeon removes a portion of the colon, it may be necessary to attach the remaining colon to the outside of the body. Creating a hole (i.e., stoma) in the abdominal wall allows waste to leave the body. As such, a colostomy bag 125 can attach to the stoma to collect waste.

As described herein, the wound cleansing apparatus includes a female snap-on ring component and a male snap-on ring base component that is connected to the female snap-on ring by a snap connection. Inside the male snap-on ring base component, there is at least one gauze positioned in such a way that it is in contact with the wound. Additionally, a draining tube is attached to the male snap-on ring base component using a latching mechanism.

The female snap-on ring component may also include a transparent cover region. The female snap-on ring component can have an air vent that can be opened or closed depending on the position. The air vent is in an open state when it is in one position, and it can be closed in another position. The female snap-on ring component may have a transparent region, and the male snap-on ring base component may also have another transparent region.

The draining tube includes a bend component near the cavity of the gauze, and the bend has an angle ranging from 20% to 40%. The latching mechanism on the male snap-on ring base component includes two members that work together to secure the draining tube in place.

The wound cleansing apparatus may also include a pair of flange components located on the exterior of the female snap-on ring component. These flanges can have adhesive on the back side, allowing them to be easily attached to a surface. The gauze has a cavity through which the draining tube is inserted.

The wound cleansing system includes the apparatus described above and further includes a fluid dispenser that is coupled to the end of the draining tube. The fluid dispenser may be a positive displacement dispenser. Both the male snap-on ring base component and the female snap-on ring cover component may be circular in shape. The system may also include an adhesive bandage on the backside of the male snap-on ring base component, which could be a waterproof band-aid. Additionally, the system may include a colostomy bag attached to the exterior of the wound cleansing apparatus.

The wound cleansing method involves applying the wound cleansing system to the wound. The system includes the female snap-on ring cover, the male snap-on ring base connected by a snap connection, a set of gauzes positioned within the male snap-on ring base in contact with the wound, and a draining tube coupled to the male snap-on ring base with a latching mechanism. The method further includes injecting fluid into the draining tube to flush the wound. The first set of gauzes may be replaced with a second set, and the latching mechanism can be engaged to secure the draining tube in place. The fluid injected into the wound is typically saline. The method may also involve disconnecting the draining tube from the male snap-on ring base component and flushing out the draining tube.

While illustrative implementations of the application have been described in detail herein, it is to be understood that the inventive concepts may be otherwise variously embodied and employed, and that the appended claims are intended to be construed to include such variations, except as limited by the prior art.

Reference throughout this specification to “one implementation” or “an implementation” means that a particular feature, structure, or characteristic described in connection with the implementation is included in at least one implementation of the present disclosure. Thus, the appearances of the phrases “in one implementation” or “in some implementations” in various places throughout this specification are not necessarily all referring to the same implementation. Furthermore, the particular features, structures, or characteristics may be combined in any suitable manner in one or more implementations.

Systems and methods describing the present disclosure have been described. It will be understood that the descriptions of some embodiments of the present disclosure do not limit the various alternative, modified, and equivalent embodiments which may be included within the spirit and scope of the present disclosure as defined by the appended claims. Furthermore, in the detailed description above, numerous specific details are set forth to provide an understanding of various embodiments of the present disclosure. However, some embodiments of the present disclosure may be practiced without these specific details. In other instances, well known methods, procedures, and components have not been described in detail so as not to unnecessarily obscure aspects of the present embodiments.

1. A wound cleansing apparatus, comprising:
a cover component;
a cover component by a coupling means;
at least one gauze positioned within an interior of the base component; and
a draining tube coupled to the base component by a latching mechanism,
wherein the latching mechanism holds the draining tube in place when the draining tube is in operation.

2. The wound cleansing apparatus of claim 1, wherein the cover component comprises a transparent cover region.

3. The wound cleansing apparatus of claim 1, wherein the base component includes an air vent.

4. The wound cleansing apparatus of claim 3, wherein the air vent is in an open state in a first position and a closed state in a second position.

5. The wound cleansing apparatus of claim 1, wherein the cover component has a first transparent region and the base component has a second transparent region.

6. The wound cleansing apparatus of claim 1, wherein the draining tube includes a first bend component at a first bend near the at least one gauze, the first bend having an angle in the range of 20% to 40%.

7. The wound cleansing apparatus of claim 6, wherein the cover includes a second bend component that mates with the first bend component of the draining tube.

8. The wound cleansing apparatus of claim 1, wherein the latching mechanism on the base component includes a first member and a second member, which together secure the draining tube in place.

9. The wound cleansing apparatus of claim 1, further comprising a pair of flange components on the exterior of the cover component.

10. The wound cleansing apparatus of claim 9, further comprising an adhesive on the backside of the flange components.

11. The wound cleansing apparatus of claim 1, wherein the at least one gauze has a cavity through which the draining tube is inserted.

12. A wound cleansing system, comprising:
a wound cleansing apparatus, comprising:
a female snap-on ring cover component;
a male snap-on ring base component coupled to the female snap-on ring cover component by a snap connection;
at least one gauze positioned within an interior of the base component; and
a fluid dispenser coupled to a first end of the draining tube.

13. The wound cleansing system of claim 12, wherein the fluid dispenser is a positive displacement dispenser.

14. The wound cleansing system of claim 12, wherein the male snap-on ring base component and the female snap-on ring component are both circular in shape.

15. The wound cleansing system of claim 12, further comprising an adhesive bandage on the backside of the male snap-on ring base component.

16. The wound cleansing system of claim 15, wherein the adhesive bandage is a waterproof band-aid.

17. The wound cleansing system of claim 12, further comprising a colostomy bag coupled to an exterior of the wound cleansing apparatus.

18. A wound cleansing method, comprising:
applying a wound cleansing system to a wound, the wound cleansing system comprising:
a wound cleansing apparatus, comprising:
a female snap-on ring cover component;
a male snap-on ring base component coupled to the female snap-on ring cover component by a snap connection;
a first set of gauzes positioned within the male snap-on ring base component, wherein the first set of gauzes is in contact with the wound; and
a draining tube coupled to the male snap-on ring base component by at least one component of the latching mechanism; and
injecting a fluid into the draining tube to flush the wound.

19. The wound cleansing method of claim 18 further comprising replacing the first set of gauzes with a second set of gauzes.

20. The wound cleansing method of claim 18 further comprising engaging the latching mechanism to secure the draining tube in place.
